(19) United States
(12) Patent Application Publication
(10) Pub. No.: US 2006/0281803 A1
(43) Pub. Date: Dec. 14, 2006

(54) Pyrazole modulators of metabotropic glutamate receptors

(76) Inventors: Craig W. Lindsley, Schwenksville, PA (US); David D. Wisnoski, Lansdale, PA (US)

Correspondence Address: MERCK AND CO., INC, P O BOX 2000, RAHWAY, NJ, 07065-0907, US

(21) Appl. No.: 10/572,772

(22) PCT Filed: Sep. 17, 2004
(86) PCT No.: PCT/US04/30482

§ 371 Date: Mar. 21, 2006

Related U.S. Application Data
(60) Provisional application No. 60/505,035, filed on Sep. 23, 2003.

(51) Int. Cl. C07D 231/50; A61K 31/4152

(52) U.S. Cl. 514/406; 548/368.4

(57) ABSTRACT

The present invention is directed to compounds which are allosteric modulators of metabotropic glutamate receptors, including the mGluR5 receptor, and which are useful in the treatment of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### BACKGROUND OF THE INVENTION

The present invention relates to compounds that allosterically modulate metabotropic glutamate receptor type 5 (mGluR5) receptor activity, affecting the sensitivity of mGluR5 receptors to agonists without affecting agonist binding or acting as agonists themselves.

The amino acid L-glutamate (referred to herein simply as glutamate) is the principal excitatory neurotransmitter in the mammalian central nervous system (CNS). Within the CNS, glutamate plays an important role in a variety of processes, including synaptic plasticity (e.g., long term potentiation (the basis of learning and memory)), motor control and sensory perception. It has become increasingly clear that a variety of neurological and psychiatric disorders are associated with alterations in the glutamatergic system. The involvement of the glutamatergic system in these pathologies makes modulation of this system an important therapeutic goal. Glutamate acts through two distinct classes of receptors. The first class, termed ionotropic glutamate receptors, are multi-subunit ligand-gated ion channels that mediate excitatory post-synaptic currents. Three types of ionotropic glutamate receptors have been identified, and while glutamate is an agonist for all three types of receptor, selective ligands have been found that activate each type. The ionotropic glutamate receptors are named for these type-selective agonists: kainate receptors, AMPA receptors and NMDA receptors.

The second class of glutamate receptor, termed metabotropic glutamate receptors (mGluRs), are G-protein coupled receptors (GPCRs) that modulate neurotransmitter release or strength of synaptic transmission, depending on whether they are located pre- or post-synaptically. The mGluRs are members of GPCR family C, and possess a large (˜560 amino acid) agonist binding domain in the amino-terminal portion of the receptor. This large agonist binding domain distinguishes family C from the other GPCR families in which the agonist binding sites are associated with the 7-strand transmembrane spanning (7TM) region or with the extracellular loops that connect the strands of this region. Eight distinct mGluRs have been identified, cloned and sequenced. These have been assigned to three groups based on structural similarity, primary signal transduction pathway, and pharmacology. Group I (mGluR1 and mGluR5) receptors are primarily located post-synaptically where they modulate ion channel activity and neuronal excitability. The group I mGluRs are coupled to Galphaq and its associated effectors such as phospholipase C. Group II (mGluR2 and mGluR3) and Group III (mGluR4, mGluR6, mGluR7 and mGluR8) receptors are primarily located pre-synaptically where they regulate release of neurotransmitters, including glutamate. Group II and III mGluRs are coupled to Galphai and its associated effectors such as adenylate cyclase.

Post-synaptic mGluRs are known to interact functionally with post-synaptic ionotropic glutamate receptors. For example, activation of mGluR5 by a selective agonist has been shown to increase the post-synaptic current carried by the NMDA receptor (Mannaioni et al. J. Neurosci. 21:5925-5934 (2001)) Therefore, modulation of mGluRs is an approach to modulating glutamatergic transmission. Published reports indicate that mGluR5 plays a role in a number of disease states including anxiety (Spooren et al. J. Pharmacol. Exp Therapeut. 295:1267-1275 (2000), Tatarczynska et al. Br. J. Pharmacol. 132:1423-1430 (2001)), schizophrenia (reviewed in Chavez-Noriega et al. Current Drug Targets: CNS & Neurological Disorders 1:261-281 (2002)), addiction to cocaine (Chiamulera et al. Nature Neurosci. 4:873-874 (2001)), Parkinson's disease (Awad et al. J. Neurosci. 20:7871-7879 (2000); Ossowska et al. Neuropharmacol. 41:413-420 (2001); Breysse et al. J. Neurosci. 22:5669-5678 (2002)) and pain (Salt and Binns Neurosci. 100:375-380 (2000), Bhave et al. Nature Neuroscience 4:417-423 (2001)). A number of mGluR agonists and antagonists have previously been described that have been developed from analogues of glutamate, quisqualate or phenylglycine (reviewed by Schoepp et al. Neuropharmacol. 38:1431-1476 (1999)). However, these ligands bind to the agonist binding site (orthosteric site) of the receptor, and therefore are not, in general, selective for an individual mGluR within a group. An alternative approach is to find compounds that modulate mGluR activity by binding to the receptor at an allosteric site; that is, a site different than the orthosteric site. Allosteric modulation can be either positive or negative, increasing or decreasing the sensitivity of the receptor to agonists. For the mGluRs, the first compounds clearly shown to interact with allosteric sites were 7-(hydroxyimino)-cyclo-propa[b]chromen-1a-carboxylate-ethyl ester (mGluR1 selective, Litschig et al. Mol Pharmacol 55:453-461 (1999)) and 2-methyl-6-(phenylethynyl)pyridine (mGluR5 selective, Gasparini et al. Neuropharmacol 38:1493-1503 (1999)). These compounds do not bind to the orthosteric binding sites of their respective receptors, but rather act as negative allosteric modulators, binding to sites in the 7TM domain of their cognate receptors to exert their inhibitory effects. Positive allosteric modulators have also been demonstrated for mGluR1 (Knoflach et al. Proc Natl Acad Sci USA 98:13402-13407 (2001)) and for mGluR5 (O'Brien et al. Mol. Pharmacol. 64:731-740 (2003)).

### SUMMARY OF THE INVENTION

The present invention is directed to compounds which are allosteric modulators of metabotropic glutamate receptors, including the mGluR5 receptor, and which are useful in the treatment of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### DETAILED DESCRIPTION OF THE INVENTION

The present invention is directed to compounds of the formula I: 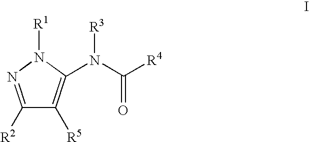
wherein:
R¹ is selected from the group consisting of:
(1) hydrogen,
(2) C₁₋₆alkyl, which is unsubstituted or substituted with halogen, hydroxyl or phenyl,
(3) C₃₋₇cycloalkyl, which is unsubstituted or substituted with halogen, hydroxyl or phenyl, and
(4) phenyl, which is unsubstituted or substituted with one or more substituents independently selected from:
   (a) —C₁₋₆alkyl,
   (b) —O—C₁₋₆alkyl,
   (c) halo,
   (d) hydroxy,
   (e) trifluoromethyl,
   (f) —OCF₃,
   (g) —CO₂R⁹,
      wherein R⁹ is independently selected from:
      (i) hydrogen,
      (ii) —C₁₋₆alkyl, which is unsubstituted or substituted with 1-6 fluoro,
      (iii) benzyl, and
      (iv) phenyl,
   (h) —NR¹⁰R¹¹,
      wherein R¹⁰ and R¹¹ are independently selected from:
      (i) hydrogen,
      (ii) —C₁₋₆alkyl, which is unsubstituted or substituted with 1-6 fluoro,
      (iii) —C₅₋₆cycloalkyl,
      (iv) benzyl,
      (v) phenyl,
      (vi) —S(O)₂—C₁₋₆alkyl,
      (vii) —S(O)₂-benzyl, and
      (viii) —S(O)₂-phenyl,
   (i) —CONR¹⁰R¹¹, and
   (j) —NO₂;
(5) heterocycle, wherein heterocycle is selected from:
benzoimidazolyl, benzimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof, which is unsubstituted or substituted with one or more substituents independently selected from:
   (a) —C₁₋₆alkyl,
   (b) —O—C₁₋₆alkyl,
   (c) halo,
   (d) hydroxy,
   (e) phenyl,
   (f) trifluoromethyl,
   (g) —OCF₃,
   (h) —CO₂R⁹,
   (i) —NR¹⁰R¹¹, and
   (j) —CONR¹⁰R¹¹;
      R² and R⁵ are independently selected from the group consisting of:
(1) hydrogen,
(2) C₁₋₆alkyl, which is unsubstituted or substituted with halogen, hydroxyl or phenyl,
(3) C₃₋₇cycloalkyl, which is unsubstituted or substituted with halogen, hydroxyl or phenyl, and
(4) phenyl, which is unsubstituted or substituted with one or more substituents independently selected from:
   (a) —C₁₋₆alkyl, which is unsubstituted or substituted with
   —NR¹⁰R¹¹,
   (b) —O—C₁₋₆alkyl,
   (c) halo,
   (d) hydroxy,
   (e) trifluoromethyl,
   (f) —OCF₃;
   (g) —CO₂R⁹,
   (h) —NR¹⁰R¹¹,
   (i) —C(O)NR¹⁰R¹¹, and
   (j) —NO₂,
(5) heterocycle, wherein heterocycle is selected from:
benzoimidazolyl, benzimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof, which is unsubstituted or substituted with one or more substituents independently selected from:
   (a) —C₁₋₆alkyl,
   (b) —O—C₁₋₆alkyl,
   (c) halo,
   (d) hydroxy,
   (e) phenyl,
   (f) trifluoromethyl,
   (g) —OCF₃;
   (h) —CO₂R⁹,
   (i) —NR¹⁰R¹¹, and
   (j) —CONR¹⁰R¹¹;
      R³ is independently selected from the group consisting of:
(1) hydrogen, and
(2) C₁₋₆alkyl;
   R⁴ is selected from the group consisting of:
(1) C₁₋₆alkyl, which is unsubstituted or substituted with halogen, hydroxyl, phenyl or heterocycle,
(2) C₃₋₇cycloalkyl, which is unsubstituted or substituted with halogen, hydroxyl or phenyl, and
(3) phenyl, which is unsubstituted or substituted with one or more substituents independently selected from:
   (a) —C₁₋₆alkyl,
   (b) —O—C₁₋₆alkyl,
   (c) halo,
   (d) hydroxy,
   (e) trifluoromethyl,
   (f) —OCF₃,
   (g) —CO₂R⁹,
   (h) —CN,
   (i) —NR¹⁰R¹¹,
   (j) —CONR¹⁰R¹¹, and
   (k) —NO₂;
(4) heterocycle, wherein heterocycle is selected from:
benzoimidazolyl, benzimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof, which is unsubstituted or substituted with one or more substituents independently selected from:
   (a) —C₁₋₆alkyl,
   (b) —O—C₁₋₆alkyl,
   (c) halo,
   (d) hydroxy,
   (e) phenyl,
   (f) trifluoromethyl,
   (g) —OCF₃,
   (h) —CO₂R⁹,
   (i) —NR¹⁰R¹¹, and
   (j) —CONR¹⁰R¹¹;
      and pharmaceutically acceptable salts thereof and individual diastereomers thereof.

An embodiment of the present invention includes compounds of the formula Ia: 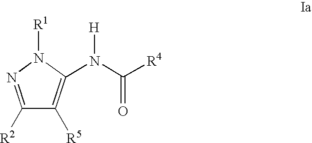
wherein
wherein R¹, R², R⁴ and R⁵ are defined herein;
and pharmaceutically acceptable salts thereof and individual enantiomers and diastereomers thereof.

An embodiment of the present invention includes compounds of the formula Ib: 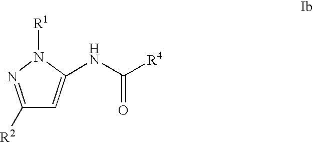
wherein R¹, R² and R⁴ are defined herein;
and pharmaceutically acceptable salts thereof and individual enantiomers and diastereomers thereof.

An embodiment of the present invention includes compounds of the formula Ic: 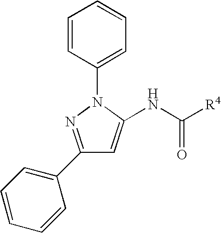
wherein R⁴ is defined herein;
and pharmaceutically acceptable salts thereof and individual enantiomers and diastereomers thereof.

A specific embodiment of the present invention includes compounds wherein R¹ is hydrogen.

Another specific embodiment of the present invention includes compounds wherein R¹ is phenyl.

A specific embodiment of the present invention includes compounds wherein R² is phenyl.

A specific embodiment of the present invention includes compounds wherein R³ is hydrogen.

An embodiment of the present invention includes compounds wherein R⁴ is phenyl, which is unsubstituted or substituted with one or more substituents independently selected from:
(a) —C₁₋₆alkyl,
(b) —O—C₁₋₆alkyl,
(c) halo,
(d) hydroxy,
(e) trifluoromethyl,
(f) —OCF₃;
(g) —CO₂—C₁₋₆alkyl,
(h) —CN,
(i) —NH₂,
(j) —NH—C₁₋₆alkyl,
(k) —CONH₂, and
(l) —CONH—C₁₋₆alkyl.

Within this embodiment, the present invention is directed to compounds wherein R⁴ is phenyl, which is unsubstituted or substituted with halo or —CN.

Within this embodiment, the present invention is directed to compounds wherein R⁴ is phenyl.

Another embodiment of the present invention includes compounds wherein R⁴ is pyridyl.

A specific embodiment of the present invention includes compounds wherein R⁵ is hydrogen.

Specific embodiments of the present invention include a compound which is selected from the group consisting of the title compounds of the Examples herein and pharmaceutically acceptable salts thereof.

The compounds of the present invention are allosteric modulators of metabotropic glutamate (mGluR) receptor function, in particular they are allosteric modulators of mGluR5 receptors. Allosteric modulation can be positive or negative. Positive allosteric modulators are referred to herein as “potentiators,” and negative allosteric modulators are referred to herein as “noncompetitive antagonists.” The term “potentiator” refers to a compound that increases or augments agonist activity, but which does not itself activate the receptor. That is, the compounds of the present invention do not appear to bind at the glutamate recognition site on the mGluR receptor, but in the presence of a sub saturating concentration of glutamate or other mGluR agonist, the compounds of the present invention increase mGluR receptor response. The present potentiators are expected to have their effect at mGluR receptors by virtue of their ability to increase the sensitivity of such receptors to glutamate or other mGluR agonists, enhancing the function of the receptors. It is recognized that the potentiators of the present invention would be expected to increase the effectiveness of glutamate and other agonists of the mGluR5 receptor. The present noncompetitive antagonists are expected to have their effect at mGluR receptors by virtue of their ability to decrease the response of such receptors to glutamate or other mGluR agonists, reducing the function of the receptors. It is recognized that the noncompetitive antagonists of the present invention would be expected to decrease the effectiveness of glutamate and other agonists of the mGluR5 receptor. Thus, the potentiators or noncompetitive antagonists of the present invention are expected to be useful in the treatment of various neurological and psychiatric disorders associated with glutamate dysfunction described to be treated herein and others that can be treated by such potentiators as are appreciated by those skilled in the art.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

As appreciated by those of skill in the art, halo or halogen as used herein are intended to include fluoro, chloro, bromo and iodo. Similarly, C₁₋₆, as in C₁₋₆alkyl is defined to identify the group as having 1, 2, 3, 4, 5 or 6 carbons in a linear or branched arrangement, such that C₁₋₈alkyl specifically includes methyl, ethyl, n-propyl, iso-propyl, n-butyl, iso-butyl, tert-butyl, pentyl, and hexyl. A group which is designated as being independently substituted with substituents may be independently substituted with multiple numbers of such substituents.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of Formula I are meant to also include the pharmaceutically acceptable salts.

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

The subject compounds are useful in a method of potentiating metabotropic glutamate receptor activity in a patient such as a mammal in need of such potentiation comprising the administration of an effective amount of the compound. The present invention is directed to the use of the compounds disclosed herein as potentiators of metabotropic glutamate receptor activity. In addition to primates, especially humans, a variety of other mammals can be treated according to the method of the present invention.

In a like manner, the subject noncompetitive antagonist compounds are useful in a method of inhibiting metabotropic glutamate receptor activity in a patient such as a mammal in need of such inhibition comprising the administration of an effective amount of the compound. The present invention is directed to the use of the compounds disclosed herein as noncompetitive antagonists of metabotropic glutamate receptor activity. In addition to primates, especially humans, a variety of other mammals can be treated according to the method of the present invention.

The present invention is further directed to a method for the manufacture of a medicament for potentiating or inhibiting metabotropic glutamate receptor activity in humans and animals comprising combining a compound of the present invention with a pharmaceutical carrier or diluent.

The subject treated in the present methods is generally a mammal, preferably a human being, male or female, in whom potentiation or inhibition of metabotropic glutamate receptor activity is desired. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with such disorders with an effective amount of the compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy of the mentioned conditions, particularly in a patient who is predisposed to such disease or disorder.

As used herein, the terms “treatment” and “treating” expressly refer to treatment of the noted conditions, to ameliorating or controlling all processes wherein there may be a slowing, interrupting, arresting, controlling, or stopping of the progression of the disorder but does not necessarily indicate a total elimination of all disorder symptoms, as well as the prevention or prophylactic therapy to retard the progression or reduce the risk of the noted conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as potentiators or noncompetitive antagonists of metabotropic glutamate receptor activity, in particular mGluR5 activity, may be demonstrated by methodology known in the art. Chinese Hamster Ovary cells transfected with human or rat mGluR5 were plated in clear bottomed assay plates for assay in a Fluorometric Plate Reader (FLIPR). The cells were loaded with a Ca²⁺-sensitive fluorescent dye (e.g. Fluo-4), and the plates were washed and placed in the FLIPR instrument. After establishment of a fluorescence baseline for 10 seconds, the compounds in the present invention were added to the cells, and the response of the cells was measured. Five minutes later, an mGluR5 agonist (e.g, glutamate, 3,5-dihydroxyphenylglycine, or quisqualate) was added to the cells, and the response of the cells was measured. Potentiation of the agonist response of mGluR5 by the compounds in the present invention was observed as an increase in response to non-maximal concentrations of agonist in the presence of compound compared to the response to agonist in the absence of compound. In a like manner, antagonism of the agonist response of mGluR5 by the compounds in the present invention was observed as a decrease in response to non-maximal concentrations of agonist in the presence of compound compared to the response to agonist in the absence of compound.

The assay described above was performed in two modalities. In the first, a range of concentrations of the present compound was added to the cells, followed by a single fixed concentration of agonist. If the compound acted as a potentiator, an EC₅₀ value for potentiation and a maximum extent of potentiation by the compound at this concentration of agonist was determined by non-linear curve fitting. If the compound acted as a noncompetitive antagonist, an IC50 value was determined by nonlinear curve fitting. In the second modality, several fixed concentrations of the present compound was added to the various wells on the plate, followed by a range of concentrations of agonist for each concentration of present compound. The EC₅₀ values for the agonist at each concentration of compound were determined by non-linear curve fitting. A decrease in the EC₅₀ value of the agonist with increasing concentrations of the present compound (a leftward shift of the agonist concentration-response curve) is an indication of the degree of mGluR5 potentiation at a given concentration of the present compound. An increase in the agonist EC₅₀ value with increasing concentrations of the present compound (a rightward shift of the agonist concentration-response curve) is an indication of the degree of mGluR5 antagonism at a given concentration of the present compound. This latter modality also demonstrates whether the present compound also affects the maximum response of mGluR5 to agonists.

In particular, the compounds of the following examples had activity in potentiating or inhibiting the mGluR5 receptor in the aforementioned assays, generally with an EC₅₀ for potentiation or an IC₅₀ for inhibition of less than about 10 μM. Preferred compounds within the present invention had activity in potentiating or inhibiting the mGluR5 receptor in the aforementioned assays with an EC₅₀ for potentiation or an IC₅₀ for inhibition of less than about 1 μM. Preferred compounds caused a change (increase or decrease) in agonist EC₅₀ value of greater than about three-fold. These compounds did not cause mGluR5 to respond in the absence of agonist, and they did not cause a significant increase in the maximal response of the mGluR5 to agonists, although the noncompetitive antagonists did cause a decrease in maximal response to agonists. These compounds acted at potentiators or noncompetitive antagonists of rat mGluR5 as well as human mGluR5. These compounds were selective for mGluR5 compared with other metabotropic glutamate receptors. Such a result is indicative of the intrinsic activity of the compounds in use as potentiators or noncompetitive antagonists of mGluR5 receptor activity.

Metabotropic glutamate receptors including the mGluR5 receptor have been implicated in a wide range of biological functions. This has suggested a potential role for these receptors in a variety of disease processes in humans or other species.

The compounds of the present invention have utility in treating a variety of neurological and psychiatric disorders associated with glutamate dysfunction, including one or more of the following conditions or diseases: cognitive disorders including dementia (associated with Alzheimer's disease, ischemia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced psychotic disorder; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, and dyskinesias [including tremor (such as rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)]; obesity, bulimia nervosa and compulsive eating disorders; pain including bone and joint pain (osteoarthritis), repetitive motion pain, dental pain, cancer pain, myofascial pain (muscular injury, fibromyalgia), perioperative pain (general surgery, gynecological), chronic pain, neuropathic pain, post-traumatic pain, trigeminal neuralgia, migraine and migraine headache; obesity or eating disorders associated with excessive food intake and complications associated therewith; attention-deficit/hyperactivity disorder; conduct disorder; mood disorders including depressive disorders, bipolar disorders, mood disorders due to a general medical condition, and substance-induced mood disorders; muscular spasms and disorders associated with muscular spasticity or weakness including tremors; urinary incontinence; amyotrophic lateral sclerosis; neuronal damage including ocular damage, retinopathy or macular degeneration of the eye, hearing loss or tinnitus; emesis, brain edema and sleep disorders including narcolepsy.

Of the disorders above, the treatment of cognitive disorders, anxiety disorders, schizophrenia or psychosis, substance use disorders and addictive behaviors, Parkinson's disease, obesity and pain are of particular importance.

In a specific embodiment, the present invention provides a method for treating cognitive disorders, comprising: administering to a patient in need thereof an effective amount of a compound of formula I. Particular cognitive disorders are dementia, delirium, amnestic disorders and age-related cognitive decline. At present, the text revision of the fourth edition of the Diagnostic and Statistical Manual of Mental Disorders (DSM-IV-TR) (2000, American Psychiatric Association, Washington D.C.) provides a diagnostic tool that includes cognitive disorders including dementia, delirium, amnestic disorders and age-related cognitive decline. As used herein, the term “cognitive disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In another specific embodiment, the present invention provides a method for treating anxiety disorders, comprising: administering to a patient in need thereof an effective amount of a compound of formula I. Particular anxiety disorders are generalized anxiety disorder, obsessive-compulsive disorder and panic attack. At present, the text revision of the fourth edition of the Diagnostic and Statistical Manual of Mental Disorders (DSM-IV-TR) (2000, American Psychiatric Association, Washington D.C.) provides a diagnostic tool that includes anxiety disorders are generalized anxiety disorder, obsessive-compulsive disorder and panic attack. As used herein, the term “anxiety disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

In another specific embodiment, the present invention provides a method for treating schizophrenia or psychosis comprising: administering to a patient in need thereof an effective amount of a compound of formula I. Particular schizophrenia or psychosis pathologies are paranoid, disorganized, catatonic or undifferentiated schizophrenia and substance-induced psychotic disorder. At present, the text revision of the fourth edition of the Diagnostic and Statistical Manual of Mental Disorders (DSM-IV-TR) (2000, American Psychiatric Association, Washington D.C.) provides a diagnostic tool that includes paranoid, disorganized, catatonic or undifferentiated schizophrenia and substance-induced psychotic disorder. As used herein, the term “schizophrenia or psychosis” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

In another specific embodiment, the present invention provides a method for treating substance-related disorders and addictive behaviors, comprising: administering to a patient in need thereof an effective amount of a compound of formula I. Particular substance-related disorders and addictive behaviors are persisting dementia, persisting amnestic disorder, psychotic disorder or anxiety disorder induced by substance abuse; and tolerance of, dependence on or withdrawal from substances of abuse. At present, the text revision of the fourth edition of the Diagnostic and Statistical Manual of Mental Disorders (DSM-IV-TR) (2000, American Psychiatric Association, Washington D.C.) provides a diagnostic tool that includes persisting dementia, persisting amnestic disorder, psychotic disorder or anxiety disorder induced by substance abuse; and tolerance of, dependence on or withdrawal from substances of abuse. As used herein, the term “substance-related disorders and addictive behaviors” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “substance-related disorders and addictive behaviors” is intended to include like disorders that are described in other diagnostic sources.

In another specific embodiment, the present invention provides a method for treating pain, comprising: administering to a patient in need thereof an effective amount of a compound of formula I. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

In another specific embodiment, the present invention provides a method for treating obesity or eating disorders associated with excessive food intake and complications associated therewith, comprising: administering to a patient in need thereof an effective amount of a compound of formula I. At present, obesity is included in the tenth edition of the International Classification of Diseases and Related Health Problems (ICD-10) (1992 World Health Organization) as a general medical condition. The text revision of the fourth edition of the Diagnostic and Statistical Manual of Mental Disorders (DSM-IV-TR) (2000, American Psychiatric Association, Washington D.C.) provides a diagnostic tool that includes obesity in the presence of psychological factors affecting medical condition. As used herein, the term “obesity or eating disorders associated with excessive food intake” includes treatment of those medical conditions and disorders described in ICD-10 and DSM-IV-TR. The skilled artisan will recognize that there are alternative nomenclatures, nosologies and classification systems for general medical conditions, and that these systems evolve with medical and scientific progress. Thus the term “obesity or eating disorders associated with excessive food intake” is intended to include like conditions and disorders that are described in other diagnostic sources.

The subject compounds are further useful in a method for the prevention, treatment, control, amelioration, or reducation of risk of the diseases, disorders and conditions noted herein.

The subject compounds are further useful in a method for the prevention, treatment, control, amelioration, or reduction of risk of the aforementioned diseases, disorders and conditions in combination with other agents, including an mGluR agonist.

The term “potentiated amount” refers to an amount of an mGluR agonist, that is, the dosage of agonist which is effective in treating the neurological and psychiatric disorders described herein when administered in combination with an effective amount of a potentiator compound of the present invention. A potentiated amount is expected to be less than the amount that is required to provided the same effect when the mGluR agonist is administered without an effective amount of a potentiator compound of the present invention.

A potentiated amount can be readily determined by the attending diagnostician, as one skilled in the art, by the use of conventional techniques and by observing results obtained under analogous circumstances. In determining a potentiated amount, the dose of an mGluR agonist to be administered in combination with a compound of formula I, a number of factors are considered by the attending diagnostician, including, but not limited to: the mGluR agonist selected to be administered, including its potency and selectivity; the compound of formula I to be coadministered; the species of mammal; its size, age, and general health; the specific disorder involved; the degree of involvement or the severity of the disorder; the response of the individual patient; the modes of administration; the bioavailability characteristics of the preparations administered; the dose regimens selected; the use of other concomitant medication; and other relevant circumstances.

A potentiated amount of an mGluR agonist to be administered in combination with an effective amount of a compound of formula I is expected to vary from about 0.1 milligram per kilogram of body weight per day (mg/kg/day) to about 100 mg/kg/day and is expected to be less than the amount that is required to provided the same effect when administered without an effective amount of a compound of formula I. Preferred amounts of a co-administered mGlu agonist are able to be determined by one skilled in the art.

The compounds of the present invention may be used in combination with one or more other drugs in the treatment, prevention, control, amelioration, or reduction of risk of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also includes therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Accordingly, the subject compounds may be used alone or in combination with other agents which are known to be beneficial in the subject indications or other drugs that affect receptors or enzymes that either increase the efficacy, safety, convenience, or reduce unwanted side effects or toxicity of the compounds of the present invention. The subject compound and the other agent may be co-administered, either in concomitant therapy or in a fixed combination.

In one embodiment, the subject compound may be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies.

In another embodiment, the subject compound may be employed in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, clonazepam, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and salts thereof, and combinations thereof, and the like, or the subject compound may be administered in conjunction with the use of physical methods such as with light therapy or electrical stimulation.

In another embodiment, the subject compound may be employed in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexol)hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In another embodiment, the subject compound may be employed in combination with acetophenazine, alentemol, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, risperidone, sulpiride, tetrabenazine, trihexyphenidyl, thioridazine, thiothixene or trifluoperazine.

In another embodiment, the subject compound may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form.

In another embodiment, the subject compound may be employed in combination with an anoretic agent such as aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; selective serotonin reuptake inhibitor (SSRI); halogenated amphetamine derivatives, including chlorphentermine, cloforex, clortermine, dexfenfluramine, fenfluramine, picilorex and sibutramine; and pharmaceutically acceptble salts thereof

In another embodiment, the subject compound may be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In another embodiment, the subject compound may be employed in combination with an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, asprin, codiene, fentanyl, ibuprofen, indomethacin, ketorolac, morphine, naproxen, phenacetin, piroxicam, a steroidal analgesic, sufentanyl, sunlindac, tenidap, and the like. Similarly, the subject compound may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of The present invention are employed. (For purposes of this application, topical application shall include mouth washes and gargles.)

The pharmaceutical composition and method of the present invention may further comprise other therapeutically active compounds as noted herein which are usually applied in the treatment of the above mentioned pathological conditions.

In the treatment of conditions which require potentiation of metabotropic glutamate receptor activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15. 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day. This dosage regimen may be adjusted to provide the optimal therapeutic response. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Abbreviations used in the description of the chemistry and in the Examples that follow are:

CH₂Cl₂ dichloromethane

DIEA diisopropylethylamine

PS-DIEA polystyrene diisopropylethylamine

PS-DMAP polystyrene 4-N,N-dimethylaminopyridine

PS-Tris polystyrene trisamine

THF tetrahydrofuran

TFA trifluoroacteic acid

MeOH methanol

Several methods for preparing the compounds of this invention are illustrated in the following Schemes and Examples. Starting materials and the requisite intermediates are in some cases commercially available, or can be prepared according to literature procedures or as illustrated herein.

The compounds of this invention may be prepared by employing reactions as shown in the following schemes, in addition to other standard manipulations that are known in the literature or exemplified in the experimental procedures. Substituent numbering as shown in the schemes does not necessarily correlate to that used in the claims and often, for clarity, a single substituent is shown attached to the compound where multiple substituents are allowed under the definitions hereinabove.

Reactions used to generate the compounds of this invention are prepared by employing reactions as shown in the Reaction Scheme I, in addition to other standard manipulations such as ester hydrolysis, cleavage of protecting groups, etc., as may be known in the literature or exemplified in the experimental procedures.

In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

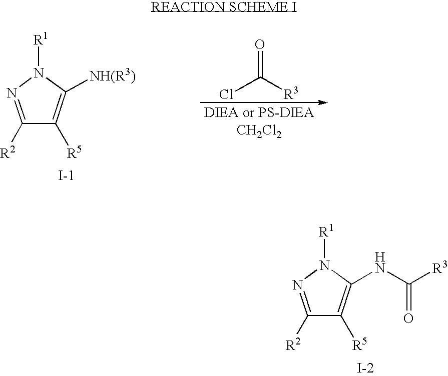

As illustrated in Reaction Scheme 1, a suitably 2-amino-1,3,4-substituted pyrazole 1-1 is acylated under standard reaction conditions to provide the corresponding amide 1-2. In this instance, all of the 2-amino-1,3,4-substituted pyrazoles 1-1 and acylating agents employed were commercialy available. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way. Particular materials employed, species and conditions are intended to be further illustrative of the invention and not limitative of the reasonable scope thereof.

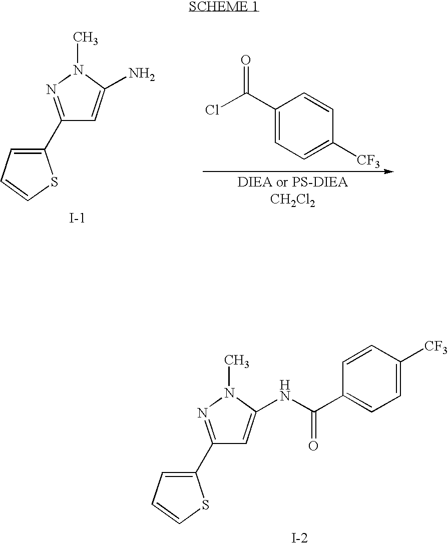

### N-[1-Methyl-3-(2-thienyl)-1H-pyrazol-5-yl]-4-(trifluoromethyl)benzamide (1-2)

To a stirred suspension of 5-amino-1-methyl-3-(thien-2-yl)pyrazole, 1-1, (30 mg, 0.168 mml), PS-DIEA (57 mg, 0.168 mol, 3.0 mmol/g) or DIEA (38 uL, 0.33 mmol) in CH₂Cl₂ (3 mL) was added p-trifluoromethylbenzoyl chloride (42 uL, 0.2 mmol). After 1 h, PS-Trisamine (96 mg, 0.33 mmol, 3.5 mmol/g) was added to scavenge the excess acid chloride. The solution was then filtered, the resin washed with CH₂Cl₂ (3 mL) and concentrated. The crude material was then purified by Mass-Guided Preparative HPLC on an Agilent 1100 system to provide 41.3 mg (70%) of the title compound, 1-2. ¹H NMR (300 MHz, DMSO-d₆): δ 7.79 (m, 2H), 7.6 (m, 1H), 7.03 (m, 2H), 6.48 (s, 1H), 3.76 (s, 3H); Analytical LCMS: single peak (214 nm) at 3.198 min (CH₃CN/H₂O/1% TFA, 4 min gradient), HRMS calc'd for C₁₆H₁₂F₃N₃OS (M+H), 352.0726; found 352.0735.

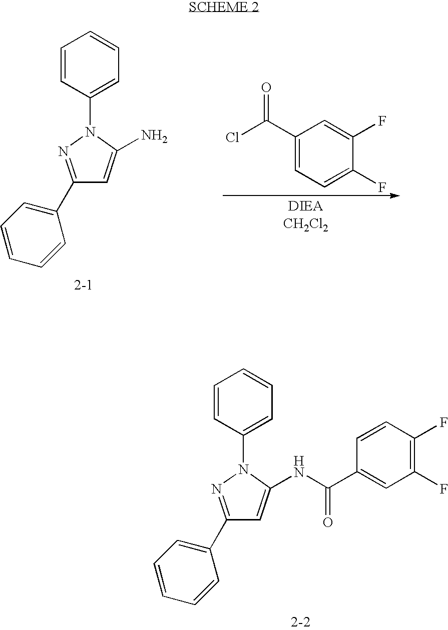

### N-(1,3-diphenyl-1H-pyrazol-5-yl)-3,4-difluorobenzamide (2-2)

To a stirred solution of 5-amino-1,3-diphenylpyrazole, 2-1, (100 mg, 0.425 mmol) in was added DIEA (150 uL, 0.85 mmol) and allowed to stir for 15 minutes followed by the addition of 3,4-difluorobenzoyl chloride (890 mg, 0.51 mmol). The solution stirred at RT until complete by TLC, and then purified by mass-guided preparative HPLC on an Agilent 1100 system. ¹H NMR (300 MHz, CDCl₃): δ 7.92 (m, 2H), 7.45 (m, 12H); Analytical LCMS: single peak (214 nm) at 3.551 min (CH₃CN/H₂O/1% TFA, 4 min gradient), HRMS calc'd for C₂₂H₁₅F₂N₃ (M+H), 376.1256; found 376.1268.

Compounds in Table 1 were synthesized as shown in Reaction Scheme 1, but substituting the appropriately substituted acid chloride as described in Scheme 1 and 2. The requisite starting materials were commercialy available, described in the literature or readily synthesized by one skilled in the art of organic synthesis.

TABLE 1

| | | MS |
|---|---|---|
| Compound | Nomenclature | M + 1 |
| 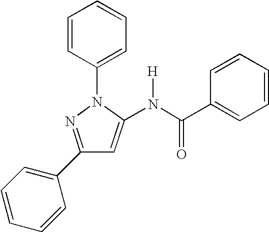 | N-(1,3-diphenyl-1H- pyrazol-5- yl)benzamide | 340.4 |
| 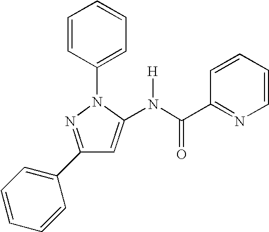 | N-(1,3-diphenyl-1H- pyrazol-5-yl)pyridine- 2-carboxamide | 341.8 |
| 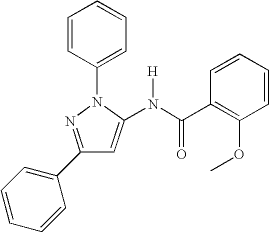 | N-(1,3-diphenyl-1H- pyrazol-5-yl)-2- methoxybenzamide | 370.4 |
| 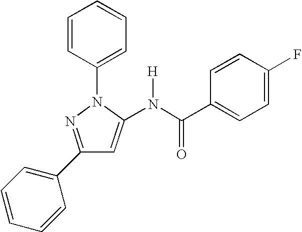 | N-(1,3-diphenyl-1H- pyrazol-5-yl)-4- fluorobenzamide | 358.3 |
| 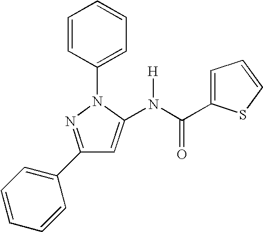 | N-(1,3-diphenyl-1H- pyrazol-5- yl)thiophene-2- carboxamide | 346.4 |
| 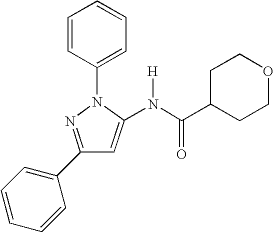 | N-(1,3-diphenyl-1H- pyrazol-5- yl)tetrahydro-2H- pyran-4-carboxamide | 348.4 |
| 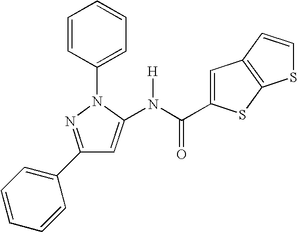 | N-(1,3-diphenyl-1H- pyrazol-5- yl)thieno[2,3- b]thiophene-2- carboxamide | 402.5 |
| 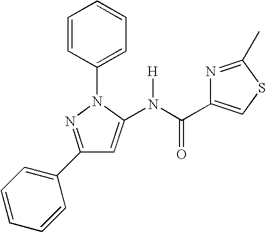 | N-(1,3-diphenyl-1H- pyrazol-5-yl)-2- methyl-1,3-thiazole-4- carboxamide | 361.4 |
| 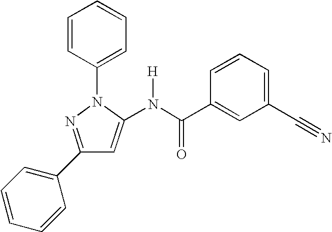 | 3-cyano-N-(1,3- diphenyl-1H-pyrazol- 5-yl)benzamide | 365.4 |
| 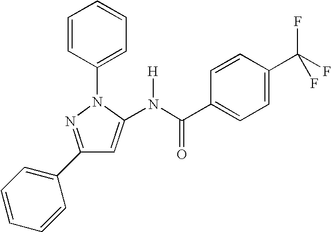 | N-(1,3-diphenyl-1H- pyrazol-5-yl)-4- (trifluoro- methyl)benzamide | 408.3 |
| 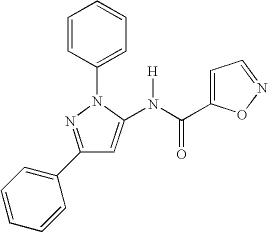 | N-(1,3-diphenyl-1H- pyrazol-5- yl)isoxazole-5- carboxamide | 331.3 |
| 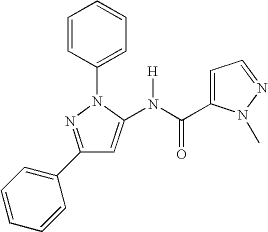 | N-(1,3-diphenyl-1H- pyrazol-5-yl)-1- methyl-1H-pyrazol-5- carboxamide | 344.3 |
| 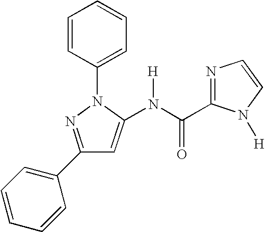 | N-(1,3-diphenyl-1H- pyrazol-5-yl)-1H- imidazole-2- carboxamide | 330.3 |
| 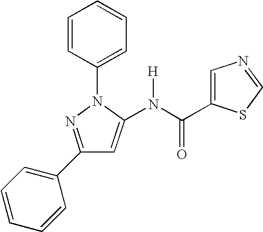 | N-(1,3-diphenyl-1H- pyrazol-5-yl)-1,3- thiazole-5- carboxamide | 347.4 |
| 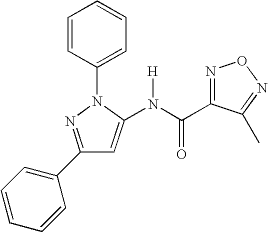 | N-(1,3-diphenyl-1H- pyrazol-5-yl)-4- methyl-1,2,5- oxadiazole-3- carboxamide | 346.3 |
| 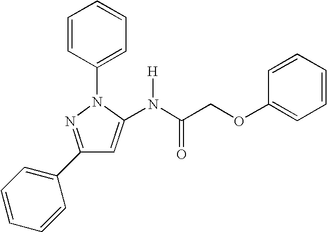 | N-(1,3-diphenyl-1H- pyrazol-5-yl)-2- phenoxyacetamide | 370.4 |
| 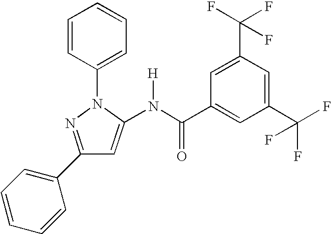 | N-(1,3-diphenyl-1H- pyrazol-5-yl)-3,5- bis(trifluoro- methyl)benzamide | 476.3 |
| 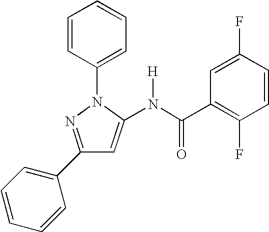 | N-(1,3-diphenyl-1H- pyrazol-5-yl)-2,5- difluorobenzamide | 376.3 |
| 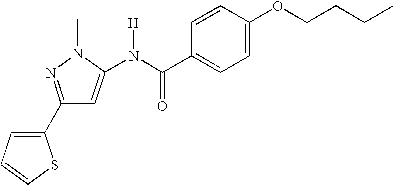 | 4-butoxy-N-[1-methyl- 3-(2-thienyl)-1H- pyrazol-5- yl]benzamide | 352.3 |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in responsiveness of the mammal being treated for any of the indications with the compounds of the invention indicated above.

What is claimed is:

1. A compound of the formula I: 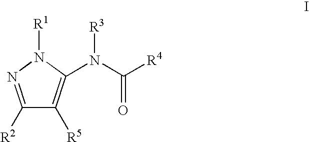
wherein:
R¹ is selected from the group consisting of:
(1) hydrogen,
(2) C₁₋₆alkyl, which is unsubstituted or substituted with halogen, hydroxyl or phenyl,
(3) C₃₋₇cycloalkyl, which is unsubstituted or substituted with halogen, hydroxyl or phenyl, and
(4) phenyl, which is unsubstituted or substituted with one or more substituents independently selected from:
(a) —C₁₋₆alkyl,
(b) —O—C₁₋₆alkyl,
(c) halo,
(d) hydroxy,
(e) trifluoromethyl,
(f) —OCF₃,
(g) —CO₂R⁹,
wherein R⁹ is independently selected from:
(i) hydrogen,
(ii) —C₁₋₆alkyl, which is unsubstituted or substituted with 1-6 fluoro,
(iii) benzyl, and
(iv) phenyl,
(h) —NR¹⁰R¹¹,
wherein R¹⁰ and R¹¹ are independently selected from:
(i) hydrogen,
(ii) —C₁₋₆alkyl, which is unsubstituted or substituted with 1-6 fluoro,
(iii) —C₅₋₆cycloalkyl,
(iv) benzyl,
(v) phenyl,
(vi) —S(O)₂—C₁₋₆alkyl,
(vii) —S(O)₂-benzyl, and
(viii) —S(O)₂-phenyl,
(i) —CONR¹⁰R¹¹, and
(j) —NO₂;
(5) heterocycle, wherein heterocycle is selected from:
benzoimidazolyl, benzimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof, which is unsubstituted or substituted with one or more substituents independently selected from:
(a) —C₁₋₆alkyl,
(b) —O—C₁₋₆alkyl,
(c) halo,
(d) hydroxy,
(e) phenyl,
(f) trifluoromethyl,
(g) —OCF₃,
(h) —CO₂R⁹,
(i) —NR¹⁰R¹¹, and
(j) —CONR¹⁰R¹¹;
R² and R⁵ are independently selected from the group consisting of:
(1) hydrogen,
(2) C₁₋₆alkyl, which is unsubstituted or substituted with halogen, hydroxyl or phenyl,
(3) C₃₋₇cycloalkyl, which is unsubstituted or substituted with halogen, hydroxyl or phenyl, and
(4) phenyl, which is unsubstituted or substituted with one or more substituents independently selected from:
(a) —C₁₋₆alkyl, which is unsubstituted or substituted with
—NR¹⁰R¹¹,
(b) —O—C₁₋₆alkyl,
(c) halo,
(d) hydroxy,
(e) trifluoromethyl,
(f) —OCF₃;
(g) —CO₂R⁹,
(h) —NR¹⁰R¹¹,
(i) —C(O)NR¹⁰R¹¹, and
(j) —NO₂,
(5) heterocycle, wherein heterocycle is selected from:
benzoimidazolyl, benzimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof, which is unsubstituted or substituted with one or more substituents independently selected from:
(a) —C₁₋₆alkyl,
(b) —O—C₁₋₆alkyl,
(c) halo,
(d) hydroxy,
(e) phenyl,
(f) trifluoromethyl,
(g) —OCF₃;
(h) —CO₂R⁹,
(i) —NR¹⁰R¹¹, and
(j) —CONR¹⁰R¹¹;
R³ is independently selected from the group consisting of:
(1) hydrogen, and
(2) C₁₋₆alkyl;
R⁴ is selected from the group consisting of:
(1) C₁₋₆alkyl, which is unsubstituted or substituted with halogen, hydroxyl, phenyl or heterocycle,
(2) C₃₋₇cycloalkyl, which is unsubstituted or substituted with halogen, hydroxyl or phenyl, and
(3) phenyl, which is unsubstituted or substituted with one or more substituents independently selected from:
(a) —C₁₋₆alkyl,
(b) —O—C₁₋₆alkyl,
(c) halo,
(d) hydroxy,
(e) trifluoromethyl,
(f) —OCF₃,
(g) —CO₂R⁹,
(h) —CN,
(i) —NR¹⁰R¹¹,
(j) —CONR¹⁰R¹¹, and
(k) —NO₂;
(4) heterocycle, wherein heterocycle is selected from:
benzoimidazolyl, benzimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof, which is unsubstituted or substituted with one or more substituents independently selected from:
(a) —C₁₋₆alkyl,
(b) —O—C₁₋₆alkyl,
(c) halo,
(d) hydroxy,
(e) phenyl,
(f) trifluoromethyl,
(g) —OCF₃,
(h) —CO₂R⁹,
(i) —NR¹⁰R¹¹, and
(j) —CONR¹⁰R¹¹;
and pharmaceutically acceptable salts thereof and individual diastereomers thereof.

2. The compound of claim 1 of the formula Ia: 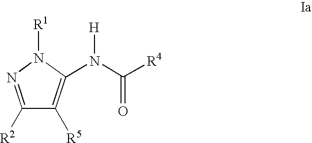
and pharmaceutically acceptable salts thereof and individual enantiomers and diastereomers thereof.

3. The compound of claim 1 of the formula Ib: 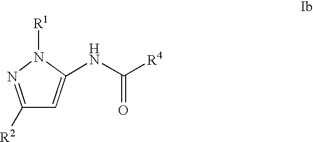
and pharmaceutically acceptable salts thereof and individual enantiomers and diastereomers thereof.

4. The compound of claim 1 of the formula Ic: 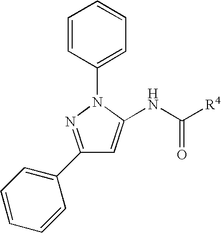
and pharmaceutically acceptable salts thereof and individual enantiomers and diastereomers thereof.

5. The compound of claim 1 wherein R¹ is hydrogen.

6. The compound of claim 1 wherein R¹ is phenyl.

7. The compound of claim 1 wherein R² is phenyl.

8. The compound of claim 1 wherein R³ is hydrogen.

9. The compound of claim 1 wherein R⁴ is phenyl, which is unsubstituted or substituted with one or more substituents independently selected from:
(a) —C₁₋₆alkyl,
(b) —O—C₁₋₆alkyl,
(c) halo,
(d) hydroxy,
(e) trifluoromethyl,
(f) —OCF₃;
(g) —CO₂—C₁₋₆alkyl,
(h) —CN,
(i) —NH₂,
(j) —NH—C₁₋₆alkyl,
(k) —CONH₂, and
(l) —CONH—C₁₋₆alkyl.

10. The compound of claim 9 wherein R⁴ is phenyl, which is unsubstituted or substituted with halo or —CN.

11. The compound of claim 10 wherein R⁴ is phenyl.

12. The compound of claim 1 wherein R⁴ is pyridyl.

13. The compound of claim 1 wherein R⁵ is hydrogen.

14. A compound which is selected from the group consisting of: 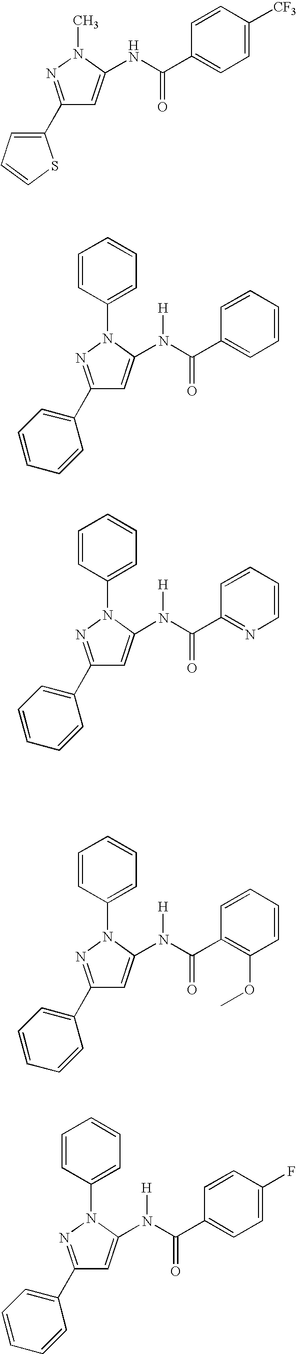 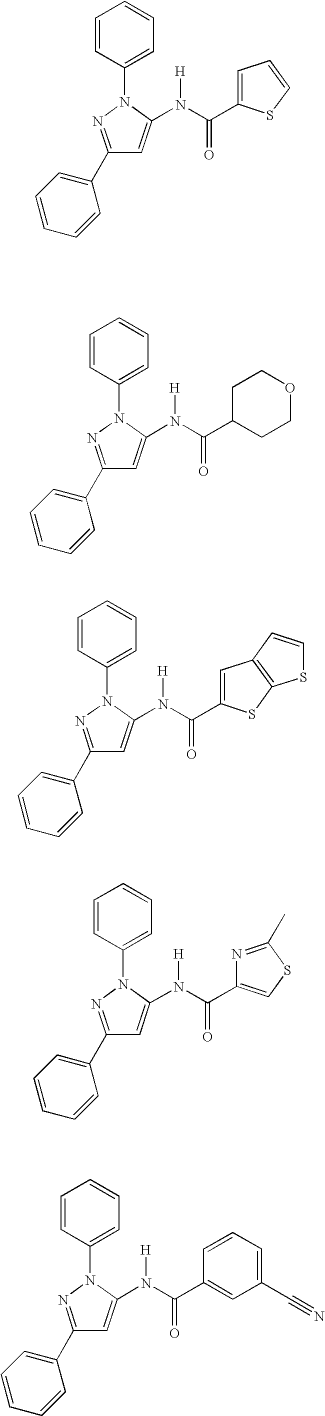 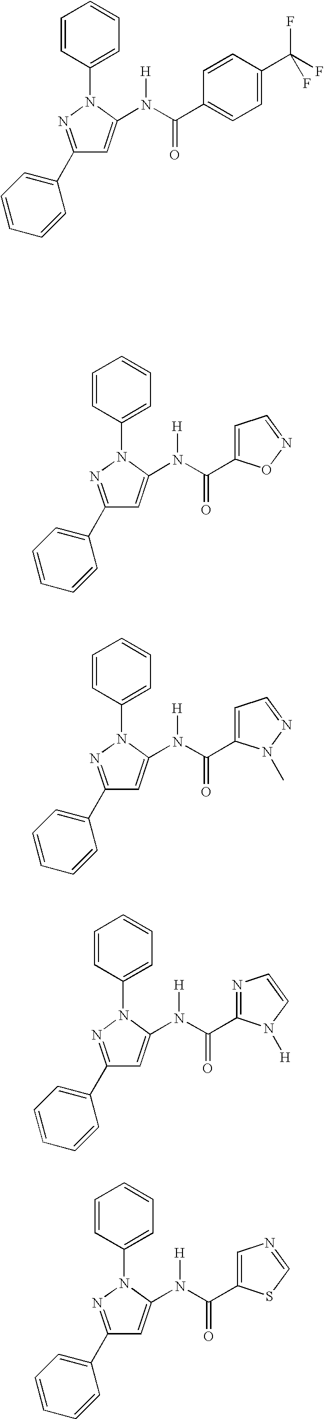 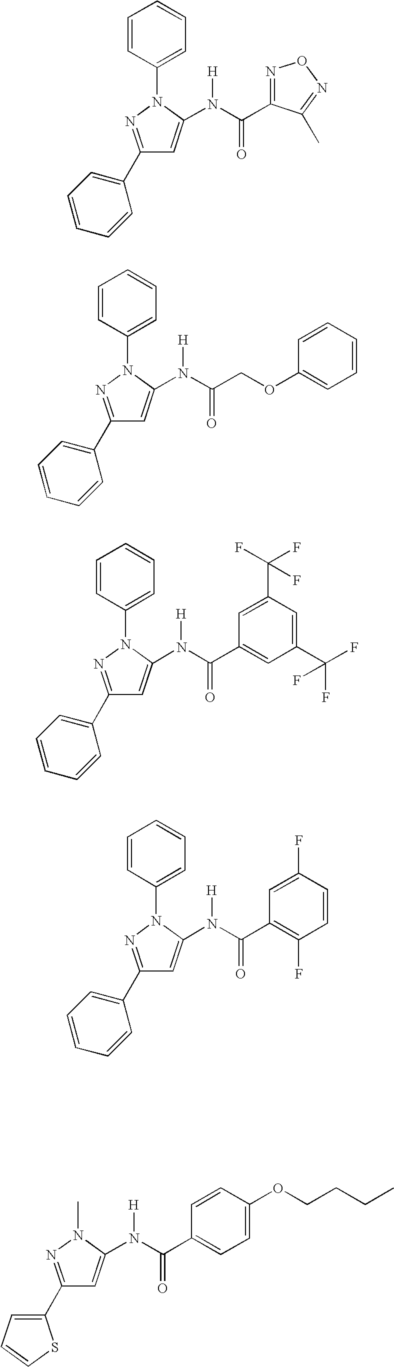
and pharmaceutically acceptable salts thereof.

15. A pharmaceutical composition which comprises an inert carrier and a compound of claim 1.

16. A method for potentiation or inhibition of metabotropic glutamate receptor activity in a mammal which comprises the administration of an effective amount of the compound of claim 1.

17. A method for the manufacture of a medicament for potentiation or inhibition of metabotorpic glutamate receptor activity in a mammal comprising combining the compound of claim 1 with a pharmaceutical carrier or diluent.

18. A method for treating a neurological and psychiatric disorders associated with glutamate dysfunction in a mammalian patient in need of such which comprises administering to the patient a therapeutically effective amount of a compound of claim 1.

19. A method for treating schizophrenia in a mammalian patient in need of such which comprises administering to the patient a therapeutically effective amount of a compound of claim 1.

20. A method for treating anxiety in a mammalian patient in need of such which comprises administering to the patient a therapeutically effective amount of a compound of claim 1.
